(19) United States
(12) Patent Application Publication
(10) Pub. No.: US 2014/0277533 A1
(43) Pub. Date: Sep. 18, 2014

(54) Internal Unloader Brace and Method

(71) Applicant: Moximed, Inc., Hayward, CA (US)

(72) Inventors: Clinton N. Slone, San Francisco, CA (US); Toru Mino, Somerville, MA (US); Anton G. Clifford, Mountain View, CA (US)

(73) Assignee: Moximed, Inc., Hayward, CA (US)

(21) Appl. No.: 14/205,897

(22) Filed: Mar. 12, 2014

Related U.S. Application Data
(60) Provisional application No. 61/779,281, filed on Mar. 13, 2013.

(51) Int. Cl. A61F 2/38

(52) U.S. Cl. CPC A61F 2/3836
USPC 623/20.24

(57) ABSTRACT

An internal unloader brace includes an elongate bending member having an intermediate portion configured to bend under resistance such that joint forces are unloaded.

This application claims priority under 35 U.S.C. §119 to U.S. Provisional App. No. 61/779,281, filed 13 Mar. 2013, the entirety of which is incorporated by reference herein.

### FIELD OF THE INVENTION

The present invention is directed towards apparatus and methods for treating tissue of a body and more particularly, towards approaches designed to reduce mechanical energy transferred between members forming a natural joint.

### BACKGROUND OF THE INVENTION

A joint is the location at which two or more bones make contact. They are constructed to allow movement and provide mechanical support, and are classified structurally and functionally. Structural classification is determined by how the bones connect to each other, while functional classification is determined by the degree of movement between the articulating bones. In practice, there is significant overlap between the two types of classifications.

There are three structural classifications of joints, namely fibrous or immovable joints, cartilaginous joints and synovial joints. Fibrous/Immovable bones are connected by dense connective tissue, consisting mainly of collagen. The fibrous joints are further divided into three types:
sutures which are found between bones of the skull;

syndesmosis which are found between long bones of the body; and
gomphosis which is a joint between the root of a tooth and the sockets in the maxilla or mandible.

Cartilaginous bones are connected entirely by cartilage (also known as “synchondroses”). Cartilaginous joints allow more movement between bones than a fibrous joint but less than the highly mobile synovial joint. An example of a cartilaginous joint is an intervertebral disc. Synovial joints have a space between the articulating bones for synovial fluid. This classification contains joints that are the most mobile of the three, and includes the knee and shoulder. These are further classified into ball and socket joints, condyloid joints, saddle joints, hinge joints, pivot joints, and gliding joints.

Joints can also be classified functionally, by the degree of mobility they allow. Synarthrosis joints permit little or no mobility. They can be categorized by how the two bones are joined together. That is, synchrondoses are joints where the two bones are connected by a piece of cartilage. Synostoses are where two bones that are initially separated eventually fuse together as a child approaches adulthood. By contrast, amphiarthrosis joints permit slight mobility. The two bone surfaces at the joint are both covered in hyaline cartilage and joined by strands of fibrocartilage. Most amphiarthrosis joints are cartilaginous.

Finally, diarthrosis joints permit a variety of movements (e.g. flexion, adduction, pronation). Only synovial joints are diarthrodial and they can be divided into six classes: 1. ball and socket—such as the shoulder or the hip and femur; 2. hinge—such as the elbow; 3. pivot—such as the radius and ulna; 4. condyloidal (or ellipsoidal)—such as the wrist between radius and carps, or knee; 5. saddle—such as the joint between carpal thumbs and metacarpals; and 6. gliding—such as between the carpals.

Synovial joints (or diarthroses, or diarthroidal joints) are the most common and most moveable type of joints in the body. As with all other joints in the body, synovial joints achieve movement at the point of contact of the articulating bones. Structural and functional differences distinguish the synovial joints from the two other types of joints in the body, with the main structural difference being the existence of a cavity between the articulating bones and the occupation of a fluid in that cavity which aids movement. The whole of a diarthrosis is contained by a ligamentous sac, the joint capsule or articular capsule. The surfaces of the two bones at the joint are covered in cartilage. The thickness of the cartilage varies with each joint, and sometimes may be of uneven thickness. Articular cartilage is multi-layered. A thin superficial layer provides a smooth surface for the two bones to slide against each other. Of all the layers, it has the highest concentration of collagen and the lowest concentration of proteoglycans, making it very resistant to shear stresses. Deeper than that is an intermediate layer, which is mechanically designed to absorb shocks and distribute the load efficiently. The deepest layer is highly calcified, and anchors the articular cartilage to the bone. In joints where the two surfaces do not fit snugly together, a meniscus or multiple folds of fibro-cartilage within the joint correct the fit, ensuring stability and the optimal distribution of load forces. The synovium is a membrane that covers all the non-cartilaginous surfaces within the joint capsule. It secretes synovial fluid into the joint, which nourishes and lubricates the articular cartilage. The synovium is separated from the capsule by a layer of cellular tissue that contains blood vessels and nerves.

Cartilage is a type of dense connective tissue and as noted above, it forms a critical part of the functionality of a body joint. It is composed of collagenous fibers and/or elastin fibers, and cells called chondrocytes, all of which are embedded in a firm gel-like ground substance called the matrix. Articular cartilage is avascular (contains no blood vessels) and nutrients are diffused through the matrix. Cartilage serves several functions, including providing a framework upon which bone deposition can begin and supplying smooth surfaces for the movement of articulating bones. Cartilage is found in many places in the body including the joints, the rib cage, the ear, the nose, the bronchial tubes and between intervertebral discs. There are three main types of cartilage: hyaline, elastic and fibrocartilage.

Chondrocytes are the only cells found in cartilage. They produce and maintain the cartilaginous matrix. Experimental evidence indicates that cells are sensitive to their mechanical (stress-strain) state, and react directly to mechanical stimuli. The biosynthetic response of chondrocytes was found to be sensitive to the frequency and amplitude of loading (Wong et al., 1999 and Kurz et al., 2001). Recent experimental studies further indicate that excessive, repetitive loading may induce cell death, and cause morphological and cellular damage, as seen in degenerative joint disease (Lucchinetti et al., 2002 and Sauerland et al., 2003). Islam et al. (2002) found that continuous cyclic hydrostatic pressure (5 MPa, 1 Hz for 4 hours) induced apoptosis in human chondrocytes derived from osteoarthritic cartilage in vitro. In contrast, cyclic, physiological-like loading was found to trigger a partial recovery of morphological and ultra-structural aspects in osteoarthritic human articular chondrocytes (Nerucci et al., 1999).

Cancellous bone (also known as trabecular, or spongy) is a type of osseous tissue which also forms an important aspect of a body joint. Cancellous bone has a low density and strength but very high surface area, that fills the inner cavity of long bones. The external layer of cancellous bone contains red bone marrow where the production of blood cellular components (known as hematopoiesis) takes place. Cancellous bone is also where most of the arteries and veins of bone organs are found. The second type of osseous tissue is known as cortical bone, forming the hard outer layer of bone organs.

Various maladies can affect the joints, one of which is arthritis. Arthritis is a group of conditions where there is damage caused to the joints of the body. Arthritis is the leading cause of disability in people over the age of 65.

There are many forms of arthritis, each of which has a different cause. Rheumatoid arthritis and psoriatic arthritis are autoimmune diseases in which the body is attacking itself. Septic arthritis is caused by joint infection. Gouty arthritis is caused by deposition of uric acid crystals in the joint that results in subsequent inflammation. The most common form of arthritis, osteoarthritis is also known as degenerative joint disease and occurs following trauma to the joint, following an infection of the joint or simply as a result of aging.

Unfortunately, all arthritides feature pain. Patterns of pain differ among the arthritides and the location. Rheumatoid arthritis is generally worse in the morning; in the early stages, patients often do not have symptoms following their morning shower.

Osteoarthritis (OA, also known as degenerative arthritis or degenerative joint disease, and sometimes referred to as “arthrosis” or “osteoarthrosis” or in more colloquial terms “wear and tear”), is a condition in which low-grade inflammation results in pain in the joints, caused by wearing of the cartilage that covers and acts as a cushion inside joints. As the bone surfaces become less well protected by cartilage, the patient experiences pain upon weight bearing, including walking and standing. Due to decreased movement because of the pain, regional muscles may atrophy, and ligaments may become more lax. OA is the most common form of arthritis.

The main symptoms of osteoarthritis is chronic pain, causing loss of mobility and often stiffness. “Pain” is generally described as a sharp ache, or a burning sensation in the associated muscles and tendons. OA can cause a crackling noise (called “crepitus”) when the affected joint is moved or touched, and patients may experience muscle spasm and contractions in the tendons. Occasionally, the joints may also be filled with fluid. Humid weather increases the pain in many patients.

OA commonly affects the hand, feet, spine, and the large weight-bearing joints, such as the hips and knees, although in theory, any joint in the body can be affected. As OA progresses, the affected joints appear larger, are stiff and painful, and usually feel worse, the more they are used and loaded throughout the day, thus distinguishing it from rheumatoid arthritis. With progression in OA, cartilage loses its viscoelastic properties and its ability to absorb load.

Generally speaking, the process of clinically detectable osteoarthritis is irreversible, and typical treatment consists of medication or other interventions that can reduce the pain of OA and thereby improve the function of the joint. According to an article entitled “Surgical approaches for osteoarthritis” by Klaus-Peter Günther, MD, over recent decades, a variety of surgical procedures have been developed with the aim of decreasing or eliminating pain and improving function in patients with advanced osteoarthritis (OA). The different approaches include preservation or restoration of articular surfaces, total joint replacement with artificial implants, and arthrodeses.

Arthrodeses are described as being reasonable alternatives for treating OA of small hand and foot joints as well as degenerative disorders of the spine, but were deemed to be rarely indicated in large weight-bearing joints such as the knee due to functional impairment of gait, cosmetic problems and further side-effects. Total joint replacement was characterized as an extremely effective treatment for severe joint disease. Moreover, recently developed joint-preserving treatment modalities were identified as having a potential to stimulate the formation of a new articular surface in the future. However, it was concluded that such techniques do not presently predictably restore a durable articular surface to an osteoarthritic joint. Thus, the correction of mechanical abnormalities by osteotomy and joint debridement are still considered as treatment options in many patients. Moreover, patients with limb malalignment, instability and intra-articular causes of mechanical dysfunction can benefit from an osteotomy to provide pain relief, with the goal being the transfer of weight-bearing forces from arthritic portions to healthier locations of a joint.

Joint replacement is one of the most common and successful operations in modern orthopedic surgery. It consists of replacing painful, arthritic, worn or diseased parts of the joint with artificial surfaces shaped in such a way as to allow joint movement. Such procedures are a last resort treatment as they are highly invasive and require substantial periods of recovery. Some forms of joint replacement are referred to as total joint replacement indicating that all joint surfaces are replaced. This contrasts with hemiarthroplasty (half arthroplasty) in which only one bone's joint surface is replaced and unicompartmental arthroplasty in which both surfaces of the knee, for example, are replaced but only on the inner or outer sides, not both. Thus, arthroplasty, as a general term, is an operative procedure of orthopedic surgery performed, in which the arthritic or dysfunctional joint surface is replaced with something better or by remodeling or realigning the joint by osteotomy or some other procedure. These procedures are also characterized by relatively long recovery times and are highly invasive procedures. The currently available therapies are not condro-protective. Previously, a popular form of arthroplasty was interpositional arthroplasty with interposition of some other tissue like skin, muscle or tendon to keep inflammatory surfaces apart or excisional arthroplasty in which the joint surface and bone was removed leaving scar tissue to fill in the gap. Other forms of arthroplasty include resection(al) arthroplasty, resurfacing arthroplasty, mold arthroplasty, cup arthroplasty, silicone replacement arthroplasty, etc. Osteotomy to restore or modify joint congruity is also an arthroplasty.

Osteotomy is a related surgical procedure involving cutting of bone to improve alignment. The goal of osteotomy is to relieve pain by equalizing forces across the joint as well as increase the lifespan of the joint. This procedure is often used in younger, more active or heavier patients. High tibial osteotomy (HTO) is associated with a decrease in pain and improved function. However, HTO does not address ligamentous instability—only mechanical alignment. HTO is associated with good early results, but results typically deteriorate over time.

Other approaches to treating osteoarthritis involve an analysis of loads that exist at a joint. Both cartilage and bone are living tissues that respond and adapt to the loads they experience. If a joint surface remains unloaded for appreciable periods of time the cartilage tends to soften and weaken. Further, as with most materials that experience structural loads, particularly cyclic structural loads, both bone and cartilage begin to show signs of failure at loads that are below their ultimate strength. However, cartilage and bone have some ability to repair themselves. There is also a level of load at which the skeleton will fail catastrophically. Bone healing research has shown that some mechanical stimulation can enhance the healing response and it is likely that the optimum regime for a cartilage/bone graft or construct will include partial/reduced from normal loading of the healing tissues.

Thus, there has been identified a need for devices which facilitate the control of load on a joint undergoing treatment or therapy, to thereby enable use of the joint within a healthy loading zone.

The present invention can satisfy these and other needs.

### SUMMARY

According to one aspect of the invention, an internal unloader brace comprises: an elongate bending member having a first end portion, a second end portion and an intermediate portion; said first end portion configured to be attached to a first anatomical member of an articulating, anatomical joint; said second end portion configured to be attached to a second anatomical member of the anatomical joint; and said intermediate portion configured to bend, under resistance, when said first end portion is attached to the first anatomical member and said second end portion is attached to the second anatomical member. When said elongate bending member is attached to a first side of the anatomical joint, said resistance to bending applies forces to the first and second anatomical members to unload a second side portion of the anatomical joint, wherein the second side is opposite the first side.

According to another aspect of the invention, an internal unloader brace comprises an elongate bending member having a first end portion, a second end portion and an intermediate portion; said first end portion being angled with respect to a longitudinal axis by a first predefined angle when said elongate bending member is in an unbiased configuration; said second end portion being angled with respect to the longitudinal axis by a second predefined angle when said elongate bending member is in an unbiased configuration. In a biased configuration, said first end portion is configured to be attached to a first anatomical member of an articulating, anatomical joint and said first end portion is closer to alignment with the longitudinal axis than when in said unbiased configuration. In said biased configuration, said second end portion is configured to be attached to a second anatomical member of an articulating, anatomical joint and said second end portion is closer to alignment with the longitudinal axis than when in said unbiased configuration. Said intermediate portion is configured to bend, under resistance, in said biased configuration and said first end portion is attached to the first anatomical member and said second end portion is attached to the second anatomical member, said bending member applies rotational forces to the first and second anatomical members through said first and second end portions, respectively.

In accordance with a further aspect of the invention, an internal unloader brace comprises an elongate bending member having a first end portion, a second end portion and an intermediate portion; said elongate member configured to be attached across an articulating, anatomical joint by attachment of said first end portion to a first anatomical member of the articulating, anatomical joint and attachment of said second end portion to a second anatomical member of the anatomical joint. Said intermediate portion applies rotational forces to locations of attachment of said first and second end portions to the first and second anatomical members, respectively. The rotational forces are applied transversely to a main plane in which articulation of the anatomical joint occurs.

In accordance with another aspect of the invention, a method for treating an articulating anatomical joint comprises attaching a first end portion of a biasing member to a first anatomical member of the anatomical joint; attaching a second end portion of the biasing member to a second anatomical member of the anatomical joint; and applying rotational forces from said biasing member to the first and second anatomical members to bias a portion of the anatomical joint by biasing portions of the first and second anatomical members away from one another.

These and other features of the invention will become apparent to those persons skilled in the art upon reading the details of the assemblies and methods as more fully described below.

### BRIEF DESCRIPTION OF THE DRAWINGS

FIG. 1 is a front view, demonstrating normal forces existing on a side of a joint.

FIG. 2 is a front view, depicting the effect an internal unloading device of the present invention has on the opposite side of the joint.

FIG. 3A illustrates an embodiment of the present invention in which a biasing member of a device is mounted to a side of the joint that is opposite of the side of the joint to be unloaded or distracted.

FIG. 3B illustrates a biasing member in a loaded configuration according to an embodiment of the present invention.

FIG. 3C illustrates the biasing member of FIG. 3B in an unloaded configuration.

FIG. 3D illustrates an end portion of a biasing member having a circular through hole according to an embodiment of the present invention.

FIG. 3E is a partial view of an embodiment of the present invention in which a biasing member of a device is mounted to a side of the joint that is opposite of the side of the joint to be unloaded or distracted.

FIG. 4 illustrates an end portion of a biasing member having an elongated or oblong through hole or slot according to an embodiment of the present invention.

FIG. 5A shows the end portion of FIG. 4 with a washer against the outer surface thereof and covering a portion of the through hole or slot according to an embodiment of the present invention.

FIG. 5B shows the end portion of FIG. 4 with a washer against the inner surface thereof and covering a portion of the through hole or slot according to an embodiment of the present invention.

FIG. 6 shows an alternative embodiment of an attachment member according to an embodiment of the present invention.

FIG. 7A is a side view and FIG. 7B is an outside surface view of an end portion of a biasing member having an alternative embodiment of an elongated through hole or slot.

### DETAILED DESCRIPTION OF EXEMPLARY EMBODIMENTS

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a screw” includes a plurality of such screws and reference to “the device” includes reference to one or more devices and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Referring now to the drawings, which are provided by way of example and not limitation, the present invention is directed towards devices and methods for treating body tissues. In applications relating to the treatment of body joints, the present invention seeks to alleviate pain associated with the function of diseased or malaligned members forming a body joint. Whereas the present invention is particularly suited to address issues associated with osteoarthritis, the energy manipulation accomplished by the present invention lends itself well to broader applications. Moreover, the present invention is particularly suited to treating synovial joints such as the knee and shoulder. However, it is also contemplated that the apparatus and method of the present invention can be employed to treat the spine facet joints and spine vertebral joints as well as other synovial and various other joints of the body such as those of the hand and feet, including those of the fingers and toes.

In one particular aspect, the present invention seeks to permit and complement the unique articulating motion of the members defining a body joint of a patient while simultaneously manipulating energy being experienced by both cartilage and osseous tissue (cancellous and cortical bone). It has been postulated that to minimize pain, off-loading/unloading or absorption of 1-40% of forces, in varying degrees, may be necessary. Variable off-loading/unloading or absorption in the range of 5-20% can be a target for certain applications. In certain specific applications, distraction is employed in the energy manipulation approach.

Conventional or surgical or minimally invasive approaches are taken to gain access to a body joint or other anatomy requiring attention. Arthroscopic approaches are thus contemplated when reasonable to both implant the energy manipulation assembly as well as to accomplish adjusting an implanted assembly. Moreover, biologically inert materials of various kinds can be employed in constructing the energy manipulation assemblies of the present invention.

In one particular approach, a biasing member is contemplated to manipulate or absorb forces between body parts on an opposite side of a joint to which the device is mounted by providing an rotational force to the body parts. Thus, a device utilizing an element or elements that can apply rotational forces to the bones that are joined by the joint may be desirable to treat afflictions such as osteoarthritis, trauma, or other pain-causing conditions in a joint.

Referring to FIGS. 1-2, forces occurring between members forming a body joint (anatomical joint) are described. The arrows 50 shown in FIG. 1 represent forces/load occurring between adjacent members 6, 7 on one side of a body joint lacking an internal unloading brace device 10 of the present invention. However, as shown in FIG. 2, in body anatomy incorporating the present invention, less forces/load are transferred to the bones and cartilage of the members defining the joint on the side that is opposite to the side that the biasing member of the device 10 is attached to. Where the body joint is treated with the described unloading devices of the present invention, a portion of the forces/load between body members is unloaded from the opposite side and taken up by the biasing member 14. Accordingly, with the internal unloading device 10 in place, less force is placed on the joint than when the assembly 10 is not present. Particularly, the opposite side of the joint is unloaded, as already noted, and as indicated by the smaller force line 56 in FIG. 2.

Although the device 10 is schematically represented as being installed on the medial side of the joint shown in FIG. 2, the present invention is not limited to such an arrangement, as device 10 can alternatively be installed on the lateral side of the joint.

FIG. 3A illustrates an embodiment of the present invention in which a biasing member 12 of device 10 is mounted to a side of the joint (i.e., to the femur 6 and tibia 7 bones) in a loaded configuration. In the loaded configuration shown in FIG. 3A, the biasing member is curved. When implanted the biasing member 12 moves from the curved configuration toward a straight configuration illustrated in FIG. 3C. This motion from the loaded (curved configuration) toward the unloaded (straight) configuration results in both motion of the bones 6, 7 away from one another and rotation of the bones 6, 7. The motion of the bones away from one another causes unloading or distraction on the same side of the joint that the device 10 is mounted to, while rotation of the ends of the device 10 cause unloading or distraction on the side of the joint that is opposite of the side of the joint where the device is implanted. The amount of unloading on each side of the joint will depend on the particular biasing member 12 and the amount of loading provided in the biasing member.

In one example, to unload or distract the medial side of a knee joint, biasing member 12 is mounted to the lateral side of that knee joint and is designed for primarily rotating and minimal bending. Conversely, to unload or distract the lateral side of a knee joint, the same biasing member 12 is mounted to the medial side of that knee joint. Alternatively, to unload the medial side of the knee joint with a medial implant, the biasing member 12 is designed for primarily elongation and minimal rotation.

FIGS. 3B and 3C illustrate biasing member 12 in a loaded configuration and an unloaded configuration, respectively. In the unloaded configuration, a first end portion 16 is angled with respect to a longitudinal axis L-L of the biasing member 12 by a predetermined acute angle 17. Angle 17 may be in the range of about twenty to about fifty degrees, typically about thirty degrees to achieve rotation of the joint and unloading of an opposite side. Likewise, the second end portion 18 is angled relative to the longitudinal axis L-L when biasing member is in an unloaded configuration as illustrated in FIG. 3C. Angle 19 typically, but not necessarily is equal to the angle 17 of the first end portion, relative to the longitudinal axis L-L, but may be different from angle 17 In either case, angle 19 may be in the range of about twenty to fifty degrees. When the biasing member 12 is designed primarily for elongation the angle 19 can be less than 20 degrees. The main body portion 14 (i.e., intermediate portion, portion that is intermediate of first and second portions 16, 18), in the unloaded configuration is substantially straight and is typically substantially aligned with the longitudinal axis L-L as shown in FIG. 3C.

In a loaded configuration, the first and second end portions are rotated to positions forming smaller angles than angles 17 and 19, respectively, relative to the longitudinal axis L-L. Typically, first and second end portions 16, 18 are substantially aligned with the longitudinal axis L-L when in a loaded configuration, as illustrated in FIG. 3B. This loading causes bending of the intermediate (main body) portion 14, as shown in FIG. 3B, such that main body portion 14 bows outwardly from longitudinal axis L-L in a direction opposite of the direction in which the end portions 16, 18 have been rotated.

Main body member is resilient, and resists bending. Accordingly, upon bending, as in FIG. 3B, the bent main body portion stores potential energy and applies forces to end portions 16, 18 in directions toward the unbiased locations of the end portions 16, 18. Main body member 14 may be made of resilient metal or metal alloy, such as, but not limited to: titanium, titanium alloys, nickel-titanium alloys, various alloys of stainless steel

In a preferred embodiment, all portions of biasing member 12 are integrally formed from metal or a metal alloy. Alternatively, one or both of first and second end components may include an elastomeric connector

When biasing member 12 is mounted to a joint, such as in a manner illustrated in FIG. 3A, main body portion 14 bends about the longitudinal axis of biasing member 12. This can be seen by comparing the loaded and unloaded illustrations of FIGS. 3B and 3C, where main body portion 14 is bent or bowed in the loaded configuration of FIG. 3B and main body portion is substantially straight, or at least significantly less bent or bowed in FIG. 3C. The elastic deformation of main body 14 that occurs in the loaded configuration shown in FIGS. 3A-3B causes biasing member 12 to apply torque in the opposite rotational directions (see arrows in FIG. 3A) to the rotational direction that effected the bending. As a result, biasing member 12, transfers force from the intermediate main body member 14 through the end portions 16 and 18 to the locations of attachment to the anatomical members 6 and 7, respectively. As applied in FIG. 3A, the forces applied are rotational forces in the clockwise direction to the femur 6 and in the counterclockwise direction to the tibia 7. This results in forces that urge medial side of the knee joint apart, when the biasing member 12 is attached to the lateral side of the knee joint as shown in the anterior view of FIG. 3A. Thus, the knee joint is partially unloaded on the medial side in this instance, reducing the amount of load that is transferred from the medial condyle of the femur to the medial condyle of the tibia during the gait cycle, relative to that which would otherwise be transferred when device 12 is not installed. In at least one embodiment, the knee joint is partially unloaded by about forty pounds on the side opposite to the side that the biasing member 12 is attached.

In general, the intermediate, bending portion 14 applies rotational forces to locations of attachment of the first and second end portions 16, 18 to the first and second anatomical members, respectively, wherein the rotational forces are applied transversely to a main plane in which articulation of the anatomical joint occurs. The biasing member 12 can thus be attached to apply rotational forces to bias first and second anatomical members away from each other on a side of the joint that is opposite a side of the joint that the biasing member 12 is attached across.

The rotational forces are applied to locations of attachment of the first and second end portions 16, 18, to the anatomy. First and second end portions 16, 18 are each provided with an opening 22 or 22′ configured and dimensioned to receive an attachment member therethrough, which is used to attach and anchor the biasing member to anatomical members forming a joint. Examples of attachment members 24 that may be used include bolts 24b and screws 24s. Opening 22 may be a round through hole 22, as shown in FIG. 3D, which is dimensioned to form a close fit with the attachment member 22 passing therethrough. In this way, the forces from the bending member 14 are efficiently transferred through the end portions 16 and 18 and attachment members 24 by maintaining the inside surfaces 16i and 18i of end portions 16 and 18 substantially normal to the longitudinal axes of the respective attachment members 24 passing through the openings 22, 22′ thereof.

The attachment locations 16a and 18a (and particularly 16a in the example of the knee) are selected to be as near to centers of rotation as possible so as to minimize any variation in length between the attachment locations over the full cycle of joint articulation (gait cycle, in the case of the knee). Further, at least one of the openings 22 may be provided as an oblong or slotted opening 22′ as illustrated in FIG. 4. Typically, only one of the end portions 16, 18 is provided with an elongated, oblong or slotted opening 22′ and the other end portion 16, 18 is provided with a round opening 22 configured and dimensioned as described above. However, both end portions 16, 18 may be provided with openings 22′. Further alternatively, one or both end portions may be provided with elastomeric connections

In FIG. 3A, the attachment members used are bolts 24, pins or bi-cortical bone screws. Nuts 24n are threaded over the distal free ends of the bolts 24 and torqued down so as to force the heads of the bolts 24 against the biasing member 12 to attach the biasing member to the anatomical members as shown in FIG. 3A. Thus, openings are established to pass completely through the anatomical members 6 and 7, from one side to the other, when using bolts 24b as attachment members 24.

FIG. 3E is a partial view of an alternative embodiment wherein screws 24s are used as the attachment members. In this case, screws 24s are provided to tap into the cortical bone 25c of the anatomical member on the opposite side of the anatomical member from the side against which the biasing member 12 is attached. Thus, attachment member 24s is passed through an opening in an end portion of the biasing member, through cortical bone 25c on the side of the anatomical member against which the biasing member 12 is to be fixed, through cancellous bone 25n, and is threaded into cortical bone 25c on the opposite side of the anatomical member, as illustrated in FIG. 3E. Upon threading/tapping the distal, threaded end of screw 24s into the cortical bone 25c on the opposite side, this forces the end portion of biasing member 12 against the anatomical member and fixes it there

Contoured spacers 28 may be provided between the inner surfaces 16i, 18i of the end portions and the outer surfaces of the anatomical members 6,7 to which the biasing member is attached. The contoured spacers 28 each have a first side generally contoured to the surface contours of the anatomical member that it is to interface with at a location where the interface will take place. The opposite side of each contoured spacer 28 is substantially perpendicular to a through hole 28h (see FIG. 3E) passing through the contoured spacer. In this way, opposite side of each contoured spacer 28 is configured to be substantially parallel with a main plane in which the anatomical joint articulates, when the first side is mounted against the first or second anatomical member. This therefore aligns the inside surfaces 16i and 18i substantially with the main plane in which the anatomical joint articulates, as can be seen in FIGS. 3A and 3E. Also, the forces applied to the anatomical member by the end portion 16, 18 are distributed by the contoured spacer 28. Likewise, contoured spacers 30 may be provided against the opposite sides of the anatomical members, between the outer surfaces of the anatomical members and mating attachment members 24n as shown in FIG. 3A. to maintain the inner surfaces of the mating attachment members 24n substantially parallel with the main plane of anatomical joint articulation and to facilitate a substantially equal application of force to the surface of the anatomical member along all radial directions from the through hole 30h.

As noted above at least one of the first and second end portions 16, 18 may be provided with an elongated or oblong opening or slot 22′ to permit the attachment member 24 to translate relative to the first or second end portion 16, 18 when the biasing member 12 is attached to the first and second anatomical members. FIG. 4 illustrates end portion 16 provided with an elongated or oblong opening or slot 22′.

FIGS. 5A and 5B illustrate inside and outside views of the end portion 16, respectively, and a washer 32 placed against the outer surface 16t or inner surface 16i, respectively, of end portion 16. Each washer 32 has a through hole 32h having an inside diameter closely fitting to an outside diameter of the attachment member 24 that passes through the washers 32 to attach end portion 16 to an anatomical member. This close fit is designed to maintain the attachment member 24 extending perpendicular to the washers 32. Thus, when washers 32 are forced against the inner and outer surfaces 16i, 16t as end portion 16 is attached to the anatomical member as described, this arrangement prevents skewing of the portion 16 out of substantial alignment with the main plane of articulation of the anatomical joint, even as attachment member 24 translates relative to elongated opening 22′, since washers 32 on both sides of slot 22′ prevent the attachment member 24 from straying from its perpendicular relationship with washers 32, and washers 32 are maintained parallel to the portion 16 by virtue of being held in contact therewith. The arrangement just described thus uses an attachment member 24, such as 24b or 24s, with a first washer 32 being slid over the attachment member 24 and against the head of the attachment member, between the head of the attachment member and the outer surface of the portion 16 or 18. A second washer 32 is slid over the attachment member 24 after sliding the portion 16 or 18 over the attachment member 24. Spacer 28 may occupy the position between the second washer 32 and the outer surface of the anatomical member, the same way as illustrated in FIG. 3A.

FIG. 6 illustrates an alternative embodiment of an attachment member 24′ that may be used in accordance with an embodiment of the present invention. Attachment member 24 may have a distal end that makes it function as a bolt 24b′ or a screw 24s′ in the same manner that the distal ends of bolts 24b and screws 24s are provided as described above. However, the proximal end of attachment member 24′ includes a washer 2432 formed integrally with (or welded to, or otherwise fixed relative to) the shaft of attachment member 24′ and extending perpendicularly to the longitudinal axis of the attachment member 24′. A predetermined length of the shaft of attachment member 24′ extends proximally of the fixed washer 2432. This predetermined length is about equal to the width of the first or second portion 16, 18. A second washer 32′ is provided to be mounted against the outside surface of the first or second portion 16, 18. This second washer has a through hole 32h′ that forms a close fit with a second attachment member 224 while allowing the shaft of the second attachment member 224 to slide therethrough. The proximal end of attachment member 24′ has an axial bore 24b′ extending therein that is internally threaded to mate with threads provided on the distal end of second attachment member 224. Thus, when first or second end portion 16, 18 is mounted over the proximal end portion 24p′ and second attachment member 224 is passed through second washer 32′ and torqued into axial threaded bore 24b′, the washers 2432 and 32′ contact opposite sides of the end portion 16 or 18 and maintain attachment member 24′ substantially perpendicular to the inside surface of end portion 16 or 18 and thus substantially perpendicular to the main plane of articulation of the anatomical joint, while allowing attachment member 24′ to translate in elongated opening 22′.

FIGS. 7A-7B illustrate another alternative arrangement that permits an attachment member 24 to translate relative to a first or second end portion 16, 18 when biasing member 12 is attached to an anatomical joint. In this embodiment, an elongated opening 22′ is provided through the first or second portion as in the previous embodiments. However, opening 22′ also extends into the first or second end portion 16, 18 to form a groove 22″ in which washer 32″ can translate along. Thus, washer 32″ is provided within the groove 22″ and can translate relative to elongated opening 22′. Attachment member 24 is maintained substantially perpendicular to the washer 32″ by the close fitting tolerances in the same manner as described above. Likewise, the groove 22″ formed in end portion 16 or 18 is only slightly thicker than the thickness of washer 32″ so as to allow washer 32″ to slide relative thereto, while at the same time maintaining washer 32″ substantially parallel to the surfaces of the groove 22″ and therefore substantially parallel to the main plane of articulation of the anatomical joint.

Further alternatively, at least one of the first and second end portions may include an elastomeric connector

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. An internal unloader brace comprising:
an elongate bending member having a first end portion, a second end portion and an intermediate portion;
said first end portion configured to be attached to a first anatomical member of an articulating, anatomical joint;
said second end portion configured to be attached to a second anatomical member of the anatomical joint; and
said intermediate portion configured to bend, under resistance, when said first end portion is attached to the first anatomical member and said second end portion is attached to the second anatomical member;
wherein when said elongate bending member is attached to a first side of the anatomical joint, said resistance to bending applies forces to the first and second anatomical members to unload a second side portion of the anatomical joint, wherein the second side is opposite the first side.

2. The internal unloader brace of claim 1, further comprising:
first and second openings through said first and second end portions, respectively; and
first and second elongate attachment members configured to pass through said first and second openings and to attach said first and second end portions to the first and second anatomical members, respectively.

3. The internal unloader brace of claim 2, wherein said first and second attachment members are configured to attach to the second side portions of the first and second anatomical members opposite to the first sides of the first and second anatomical members against which the elongate bending member is attached.

4. The internal unloader brace of claim 3, wherein said first and second attachment members are bolts, said brace further comprising first and second threaded mating members adapted to threadably engage with threaded ends of said first and second bolts, and against external surfaces of the first and second anatomical members.

5. The internal unloader brace of claim 3, wherein said first and second attachment members are screws, said first and second screws having threaded distal ends adapted to threadably anchor into cortical bone of the second side portions of the first and second anatomical members.

6. The internal unloader brace of claim 1, further comprising first and second contoured spacers, said first and second contoured spacers each having a first side generally contoured to surface contours of the first and second anatomical members at locations where the first and second end portions are to be attached; and
said first and second contoured spacers each having a second side configured to be substantially parallel with a main plane in which the anatomical joint articulates, when said first sides are mounted against the first and second anatomical members.

7. The internal unloader brace of claim 2, wherein at least one of said first and second openings comprises a slot, wherein said slot permits said first or second attachment member to translate relative to said first or second end portion when said first or second end portion is attached to the first or second anatomical member, respectively.

8. The internal unloader brace of claim 7, further comprising a washer, said washer having a through hole having an inside diameter closely fitting to an outside diameter of said first or second attachment member to maintain said first or second attachment member perpendicular to said washer, said washer configured to be mounted on said first or second attachment member and over said slot on an external side thereof, when said first or second end portion is attached to said first or second anatomical member.

9. The internal unloader brace of claim 7, wherein at least one of said attachment members comprises a washer extending perpendicularly from a shaft thereof, and a proximal end portion of said shaft extending proximally of said washer.

10. The internal unloader brace of claim 9, further comprising a second washer configured to be attached to a proximal end of said at least one of said attachment members.

11. The internal unloader brace of claim 9, wherein said at least one of said attachment members comprises an internally threaded bore extending into a proximal end of said at least one of said attachment members; said internal unloader brace further comprising a second attachment member configured to be threaded into said internally threaded bore.

12. The internal unloader brace of claim 1, wherein at least one of said first and second end portions comprises an elastomeric connector.

13. The internal unloader brace of claim 1, wherein said intermediate portion is metal.

14. The internal unloader brace of claim 1, wherein said elongate bending member is metal.

15. The internal unloader brace of claim 4, further comprising:
first and second contoured back spacers, said first and second contoured back spacers each having a first side generally contoured to surface contours of the first and second anatomical members at locations where said first and second threaded mating members are to be attached; and
wherein said first and second contoured back spacers each have a second side configured to be substantially parallel with a main plane in which the anatomical joint articulates when said first sides are mounted against the first and second anatomical members.

16. The internal unloader brace of claim 1, wherein the articulating, anatomical joint is a knee joint.

17. An internal unloader brace comprising:
an elongate bending member having a first end portion, a second end portion and an intermediate portion;
said first end portion being angled with respect to a longitudinal axis by a first predefined angle when said elongate bending member is in an unbiased configuration;
said second end portion being angled with respect to the longitudinal axis by a second predefined angle when said elongate bending member is in an unbiased configuration;
wherein, in a biased configuration, said first end portion is configured to be attached to a first anatomical member of an articulating, anatomical joint and said first end portion is closer to alignment with the longitudinal axis than when in said unbiased configuration;
wherein, in said biased configuration, said second end portion is configured to be attached to a second anatomical member of an articulating, anatomical joint and said second end portion is closer to alignment with the longitudinal axis than when in said unbiased configuration;
said intermediate portion configured to bend, under resistance, in said biased configuration; and
when said first end portion is attached to the first anatomical member and said second end portion is attached to the second anatomical member, said bending member applies rotational forces to the first and second anatomical members through said first and second end portions, respectively.

18. The internal unloader brace of claim 17, further comprising:
first and second openings through said first and second end portions, respectively; and
first and second elongate attachment members configured to pass through said first and second openings and to attach said first and second end portions to the first and second anatomical members, respectively.

19. The internal unloader brace of claim 18, wherein at least one of said first and second openings comprises a slot, wherein said slot permits said first or second attachment member to translate relative to said first or second end portion when said first or second end portion is attached to the first or second anatomical member, respectively.

20. An internal unloader brace comprising:
an elongate bending member having a first end portion, a second end portion and an intermediate portion;
said elongate member configured to be attached across an articulating, anatomical joint by attachment of said first end portion to a first anatomical member of the articulating, anatomical joint and attachment of said second end portion to a second anatomical member of the anatomical joint; and
wherein said intermediate portion applies rotational forces to locations of attachment of said first and second end portions to the first and second anatomical members, respectively; and
wherein the rotational forces are applied transversely to a main plane in which articulation of the anatomical joint occurs.

21. The internal unloader brace of claim 20, wherein the rotational forces are applied to bias the first and second anatomical members away from each other on a side of the joint that is opposite a side of the joint that the elongate member is attached across.

22. A method for treating an articulating anatomical joint, said method comprising:
attaching a first end portion of a biasing member to a first anatomical member of the anatomical joint; and
attaching a second end portion of the biasing member to a second anatomical member of the anatomical joint; and
applying rotational forces from said biasing member to the first and second anatomical members to bias a portion of the anatomical joint by biasing portions of the first and second anatomical members away from one another.

23. The method of claim 22, where the application of rotational forces biases apart a portion of the anatomical joint closer to a side of the joint opposite the side of the joint to which the biasing member is attached.

24. The method of claim 22, wherein the rotational forces are applied transverse to a main plane in which articulation of the anatomical joint occurs.

25. The method of claim 22, wherein an intermediate portion of said biasing member bends under resistance upon attachment of said first and second end portions to the first and second anatomical members; and
wherein said rotational forces are applied by the bending resistance of said intermediate portion through said first and second end portions.

26. The method of claim 23, wherein after implantation, the biasing member moves from a biased configuration to a less biased configuration by straightening, and wherein straightening of the biasing member biases apart a portion of the anatomical joint on the side to which the biasing member is attached.

27. The method of claim 22, wherein said anatomical joint is selected from the group consisting of a finger, a toe, and a knee.
